(19) United States
(12) Patent Application Publication
(10) Pub. No.: US 2025/0125025 A1
(43) Pub. Date: Apr. 17, 2025

(54) Health Data Management: Systems and Methods for Self-governance and Monetization of Health Data

(71) Applicant: DrDoRo Institute, LLC, Spring, TX (US)

(72) Inventors: Doreen Rosenstrauch, Spring, TX (US); Atul Gupta, Mississauga (CA)

(73) Assignee: DrDoRo Institute, LLC, Spring, TX (US)

(21) Appl. No.: 18/486,343

(22) Filed: Oct. 13, 2023

(51) Int. Cl. G16H 10/60; G06Q 30/0208

(52) U.S. Cl. CPC G16H 10/60; G06Q 30/0208

(57) ABSTRACT

Provided is a method for self-governance, managing, controlling, sharing and monetization of health data for human and animal entities. The method includes a user having access and control of health data for the entity as in some cases such as minors (as per prevailing age threshold in jurisdiction), children and animals being pets and livestock. The method further includes health data being shared and monetized for healthcare facilities and research organizations. The method further includes donation of health data for research after the lifetime of the entity. The method further includes methods for data protection of the health data.

### BACKGROUND OF THE INVENTION

### Field of Invention

The present invention is an application that relates to systems and methods for entities to self-govern and monetize their health data under the premise of Digital Health Data Management.

### Description of Related Art

To establish related art of self-governance and monetization of health data an advanced patent search with the Patent Public Search tool of the USPTO utilizing aforementioned key words Boolean operator AND was performed rendering the patent US20120069131 A1. While mentioning these key words, the patent, however, does not focus on self-governing and monetizing of one's own health data.

In addition, scholarly publications were researched under the same premises revealing Travieso's opinion that businesses can monetize their own data by opening these to third parties or can enhance its proprietary data to improve its services (Travieso, 2021). However, the author does not mention that patients could monetize their own health data. Furthermore, Prainsack et al., hold an opposing view of the current invention by providing reasons as to why paying individual people for their health data is a bad idea (Prainsack, 2022). In a literature review authors examine developments in privacy and data ownership in mobile health technologies and emphasize the need for improved access to control strategies (Galvin, 2020). However, the literature review neither addresses the self-governance of an individual's own data nor the monetization of such.

### PATENTS

US20120069131 A1

### PUBLICATIONS

Galvin H K, DeMuro P R. Developments in Privacy and Data Ownership in Mobile Health Technologies, 2016-2019. Yearb Med Inform. 2020 August; 29 (1): 32-43. doi: 10.1055/s-0040-1701987. Epub 2020 Aug. 21. PMID: 32823298; PMCID: PMC7442507.
Prainsack, B., Forgó, N. Why paying individual people for their health data is a bad idea. Nat Med 28, 1989-1991 (2022). doi.org/10.1038/s41591-022-01955-4
Travieso, F., Healthcare Data: A Holy Grail for Data Monetisation, Health Management, Volume 21—Issue 7, 2021

### BACKGROUND

The present disclosure relates to digital health data management, and more particularly for entities to self-govern and monetize their own health data.

In today's world of healthcare management, all the health data is owned, managed and governed by healthcare providers, facilities and professionals. Even though the health data originates and belongs to the entity who could be an individual patient treated, attended or consulted in healthcare facility or an animal pet or livestock treated in veterinary care facility. Often, disconnected health data is observed when healthcare facilities initiate similar lab requests and other diagnostics where the results from previous treatments are not readily available, accessible or in the desired formats in timely way.

### BRIEF SUMMARY OF THE INVENTION

Embodiments of the present disclosure include systems and methods with their proprietary design, methods and applications to self-govern, manage, control, share and monetize health data for human and animal entities. The health data includes personal, medical, health data for the entity as collected from healthcare facilities, physicians, surgeons, sensors directly attached or in vicinity of the patient, socio-economic data, biometric data, and personal data. The health data for the user can be stored and accessed using many methods such as secured data-store or federated links accessed in real-time as per entity preferences.

Unlike current techniques in healthcare management, the above-described embodiments will expedite data availability and bridge the gap between healthcare providers and patients, by
Avoiding Digital data-divide and bring all health data into single authoritative and trusted source to be used by all facilities world-wide;
Collecting, securing, protecting and facilitating data sources for multiple health purposes;
Not limited health data to healthcare facilities;
Collecting data from modern technology devices, sensors, biometrics, socio-economic and environment data gatherers.
Unlike current techniques in healthcare management including existing systems and technology which do not provide an efficient means for entities to directly control, monetize and aggregate their health data from healthcare facilities, and variety of sensors, the above-described embodiments all of entities health data compiled in one container with their and full control ability to monetize it.

Furthermore, these embodiments provide a new set of systems and methods for self-governance and monetization of entity's health data and gives back the health data ownership back to the entity.

The above summary is not intended to describe each illustrated embodiment or every implementation of the present disclosure.

### BRIEF DESCRIPTION OF DRAWINGS

The drawings included in the present disclosure are incorporated into, and form part of, the specification. They illustrate embodiments of the present disclosure and, along with the description, explain the principles of the disclosure. The drawings are only illustrative of typical embodiments and do not limit the disclosure.

FIG. 1 illustrates a block diagram of an example environment and framework in which illustrative embodiments of the present disclosure may be implemented. This drawing shows some examples of the types of data collected for the entity during the initial configuration and then life-long updates are collected and transferred using methods in this invention. The example methods are data-store method and federated-links method. The resulting Entity Health Data is the single authoritative, trusted, self-governed, secured, controlled, protected, and managed data.

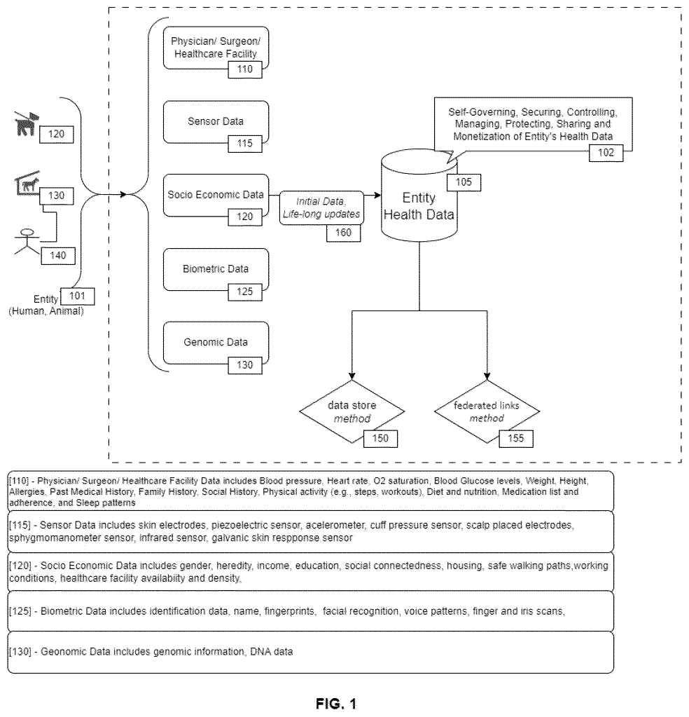

FIG. 2 illustrates a use case and data flow for the healthcare services using the applicable systems and methods of this invention for the given scenario, in accordance with embodiments of the present disclosure. This drawing shows a use case where entity feels ill while on cruise and needs immediate healthcare services away from ground and have limited on-board cruise medical team and expertise. The on-board cruise medical team access QR-code on entity's body, which is one of the methods for secure data-access. The tokenized data of the entity is sent to ground paramedic's team for consultation. Once the entity is with the ground team, physician/surgeon can access the trusted authoritative entity's health data to provide healthcare services. The Entity health data is subsequently updated with all the interactions during this use case so the single authoritative data source is maintained.

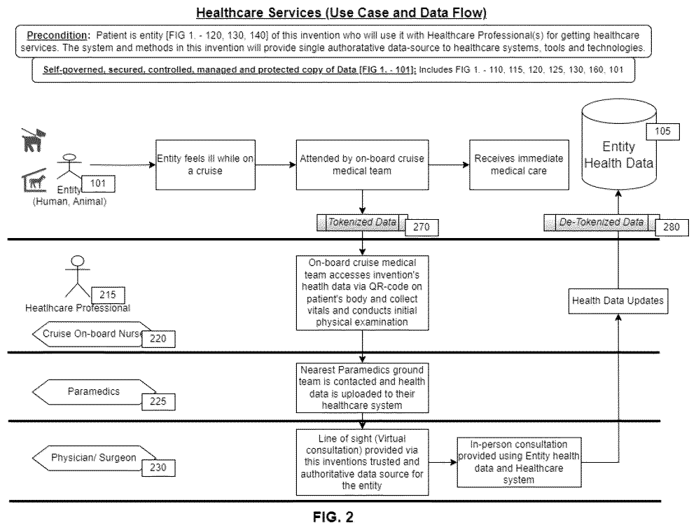

FIG. 3 illustrates a use case and data flow for using the health data in research organizations as applicable for research purposes during the entity's lifetime, in accordance with embodiments of the present disclosure. This drawing shows the Entity Health data shared for active and passive research activities to the research organizations. Under active research, monetization and health data updates are received by the Entity whereas under passive research only the monetization is received by the Entity.

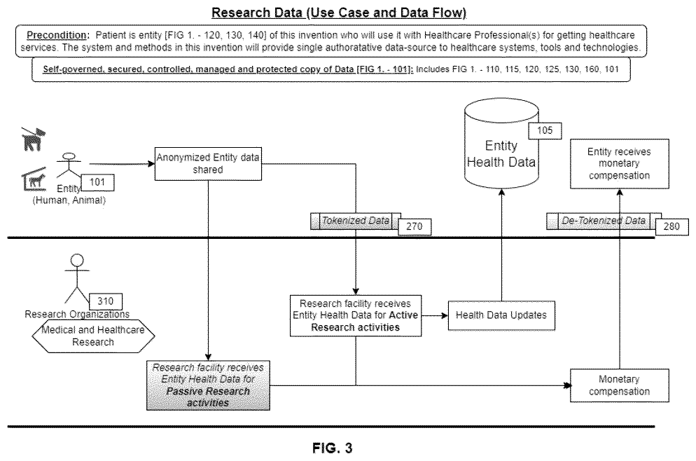

FIG. 4 illustrates a use case and data flow for using the health data in research organizations as applicable for research purposes after entity's lifetime, in accordance with embodiments of the present disclosure. This drawing shows the Entity Health data shared for active and passive research activities as per Entity's Will document disclosure and executed by the Will's executor.

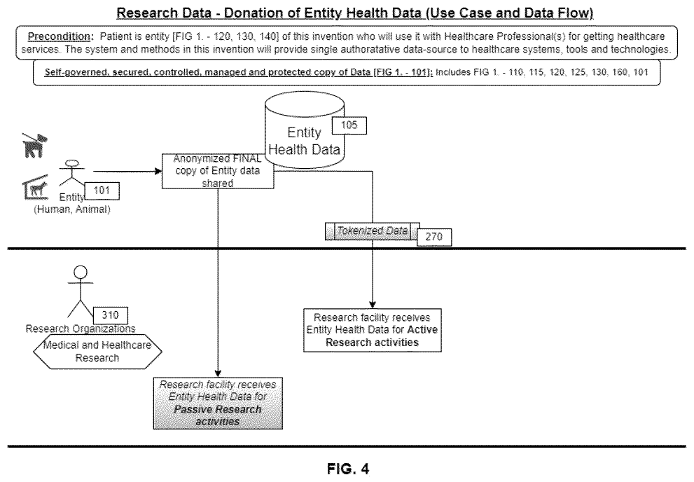

FIG. 5 illustrates a use case and data flow for requesting and engaging data protection policies, in accordance with embodiments of the present disclosure. This drawing shows examples of methods which can be used for data being dropped, deleted, permissions revoked under federated links method and also un-learning of health data when consumed by machine learning algorithms in AI methods. While the embodiments described herein are amenable to various modifications and alternative forms, specifics thereof have been shown by way of example in the drawings and will be described in detail. It should be understood, however, that the embodiments described are not to be taken in a limiting sense. On the contrary, the intention is to cover all modifications, equivalents, and alternatives falling within the spirit and scope of the invention.

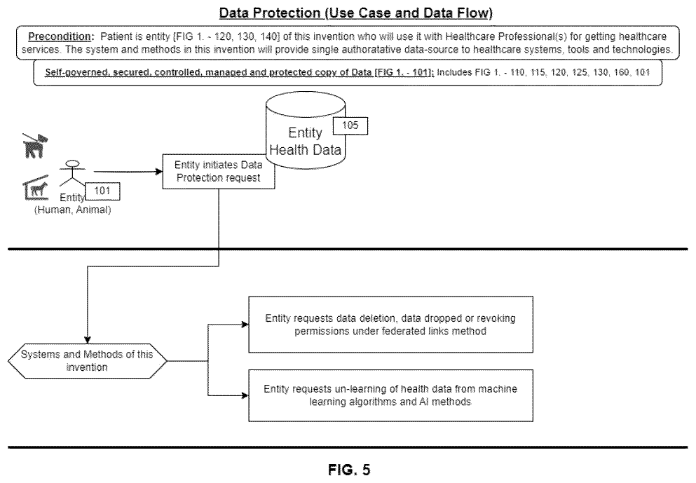

### DETAILED DESCRIPTION OF THE INVENTION

Aspects of the present disclosure relate generally to the field of digital health data management, and more particularly to self-governance and monetization of health data. While the present disclosure is not necessarily limited to such applications, various aspects of the disclosure may be appreciated though a discussion of various examples using this context.

Users currently have no access to their own health data and in current techniques, healthcare facilities, companies and research organizations use health data at their privilege and extent is often not known by the data owners. This invention will dis-mantle those data silos and copies of data existing with healthcare facilities.

This invention in the field of Digital Health Data Management is to govern, manage health, personal and medical data by collecting, storing, protecting, controlling, securing, sharing, and monetizing trusted personal health data from the data-store created and managed by the user/entity.

Embodiments of the current disclosure have the trusted personal health container encapsulating all health data by either storing and securing the health data or storing the federated links of the entity's health data. It will authorize, control and administer the sharing and distribution of data by providing it to healthcare services providers as well as to the research organizations, companies for the prospective monetization of health data. The health data will be single authoritative source of unaltered entity's personal, health, and biometric data along with socio-economic, financial and environmental data associated with the entity's health.

Embodiments of the current disclosure provides a unified container owned by the entity, governed by the user, containing all their health-related data in one place under their ownership and management.

The systems and methods will provide protection and privacy capabilities including transparency, notifications and methods to delete, un-learn in case of AI and machine learning algorithms, revoke all permissions and drop all copies of entity's health data from all identified data sources as captured and retained.

Embodiments of the current disclosure will have design and methods, and eventually a software application for web, mobile devices for the subscriber to administer, control, secure and manage the health data.

This invention will make entity's health data portable, pluggable, and connectable to third party systems and applications for the providing healthcare services and research purposes.

The different flavors of entity's health data collected will include
personal health;
genomic data, data of Deoxyribonucleic acid (DNA) sequences encoding for health and disease of an individual;
biometric data;
health data from healthcare facilities;
sensor's data where entity has been in contact with devices intentionally or unintentionally;
socio-economic, financial and environmental data related to entity's health;
Personal Identifiable Information (PII), Personal Health Identifiers (PHI), and may use formats as per HL7, FHIR, DICOM specifications and governing EHR/EMR standards and/or proprietary specifications to meet and suit the requirements of health data ecosystem;
secured data as per entity's consent, privacy and security methods and protection controls;

Embodiments of the current disclosure will have methods to govern data by mutually known and recognized policies and prevailing jurisdictional legislation, guidelines and statutory policies.

Embodiments of the current disclosure will have multiple methods for storing the collected data and will be chosen by users and entities.

The first proposed method is store method and second is federated links method. The store method will offer locally governed and secured storage for entity's health data. The federated links method will offer a compiled inventory of federated data sources as provided by the entity to generate single unified data store for the entity. Store method Vs Federated links method has their own advantages and disadvantages.

Some of the highlighted ones may include—
store method will have comparable less latency, single and relatively more secure authoritative store;
federated links method will have varied latency as it fetches data from multiple subscriber/party provided links with varied security. As the platform gains maturity, the federated links option may migrate and bring the data into store method for storing purposes.

Embodiments of the current disclosure will have design, methods and associated applications to collect health data. The methods will enable the collection of implicit and explicit data from health sensors, healthcare facilities including but not limited to wearable devices, skin sensors, and external sensors attached to the entity's body or implanted devices. The entity will have complete control over storing and un-storing their personal data and health related data. It further allows the entity to selectively share their data with healthcare professionals and providers such as physicians, surgeons, nurse practitioners and healthcare facilities, at no cost, to assist in the entity's healthcare services.

Embodiments of the current disclosure will have methods whereby entity may choose to monetize their health data for Research initiatives and data driven technologies such as Artificial Intelligence (AI), analytics and other decision making purposes. Entity may share and sell full or partial data to third parties, such as researchers, pharmaceutical companies, health tech companies, and others interested in subscribers and/or party's health data. The individual's personal identifiable information (PII), personal health identifiers (PHI) will be tokenized when data is shared outside of this invention's health data container and de-tokenized when brought back. Only anonymized data is shared outside of this invention's health data container for monetization purposes.

Embodiments of the current disclosure will have entity control, full data protection and privacy maintained and user/entity can request to drop/delete their data. The methods herein will provide transparency, notifications and facilitate deletion, revocation of permissions, un-learning from machine learning algorithms and dropping all copies of entity's data from all identified data sources as captured and retained.

Embodiments of the current disclosure will have following glossary included for the purposes of this invention—
Invention: For the purposes of governing, monetization and managing entity's health, personal and medical data relating to their health.
Entity: A person/group whose health data is stored, governed and monetized using prevailing systems and methods of this invention.
User: An individual, human person, including children and/or minors (as per prevailing age threshold in jurisdiction), animals_as pets and/or livestock, and they may be represented by individual(s) in the capacity of custodian e.g. in case of minors, children, pets and livestock, etc.
Self-governance: Ability of a person or an individual to exercise all necessary functions of regulation without intervention from an external authority.
Personal identifiable information (PII): Any representation of information that permits the identity of an individual to whom the information applies to be reasonably inferred by either direct or indirect means.
Personal health identifiers (PHI): individually identifiable health information including
   Protected health information
   Education records;
   Employment records held by an employer;
Tokenized: Data is tokenized when associated with hash generated tokens to hide PII, PHI and other sensitive data.
De-tokenized: The hash tokens are de-tokenized so data can be brought back and merged with relevant data entities in the source data store.
Anonymized: Data is considered anonymized when data is in usable format for using it for analytics, aggregations, and running machine learning and AI algorithms. The anonymization techniques and algorithms can be selected as per the system and capability requirements.
Persona: A persona is someone who is recognized by law as having legal rights and duties. A legal entity is a business organization that is legally permitted to enter into contracts.

The invention claimed is:

1. A method comprising for independent claim 1 is:
a distinctive method for entity to own, govern, secure, control and manage their health data from various sources such as healthcare facilities, sensors, socio-economic data generators, biometric devices comprising of receiving data from:
Multiple data sources including sensors connected by contact or virtually to body, biometric devices and healthcare facilities where entity has been attended or has been provided consultation;
Socio-economic data generators, financial applications, and environmental data gatherers where entity has been attended or has been in close contact or in their vicinity environment.

2. The method of claim 2 dependent on independent claim 1, comprising of:
Collecting and storing the incoming health data in secured data-store or federated links accessed in real-time as per entity preferences.

3. The method of claim 3 dependent on independent claim 1, comprising of:
Governing, securing, controlling and protecting entity's health data.

4. The method of claim 4 dependent on independent claim 1, comprising of:
Sharing, sending and receiving health data from and to healthcare professionals, providers and organizations ‘on-demand basis’;
Methods for the purposes of:
Tokenizing the requested health data;
Sharing the health data via secure channel(s);
Receiving back the usage statistics, data updates and other related data;
Detokenizing and merging back with primary data store.

5. The method of claim 5 dependent on independent claim 1, comprising of:
Sharing, sending and receiving health data from and to research organizations;
Receiving monetary compensation for the use of shared data from the research organizations. The research oriented third parties may include healthcare professionals, healthcare organizations, researchers, health technology companies, pharmaceutical companies, insurance companies, marketing agencies, social media entities, contract research organizations (CRO), and other related parties;
Methods for the purposes of:
Tokenizing the requested health data;
Sharing the data via secure channel(s);
Entity receiving the monetary benefits from research oriented third parties;
Entity receiving back the usage statistics, data updates and other related data.

6. The method of claim 6 dependent on independent claim 1, comprising of:
Methods to facilitate deletion, un-learning and dropping all copies of entity's health data from all identified data sources as captured and retained;
Methods for the purposes of:
Identifying entity, user (if applicable) and their health data;
Initiate and notify the data protection request;
Deleting, dropping, revoking permissions on entity's health data;
Un-learning entity's health data from machine learning algorithms and AI systems;
Completion and notification to the entity of the data protection request;
Embodiments of the current disclosure also include glossary of following terms for better understanding the context in which these are being used.
